(19) United States
(12) Patent Application Publication
(10) Pub. No.: US 2016/0332006 A1
(43) Pub. Date: Nov. 17, 2016

(54) Motion Mechanisms for Ultrasound Transducer Modules

(71) Applicant: Ardent Sound, Inc., Mesa, AZ (US)

(72) Inventors: Michael H. Slayton, Tempe, AZ (US); Peter G. Barthe, Phoenix, AZ (US)

(21) Appl. No.: 15/095,566

(22) Filed: Apr. 11, 2016

Related U.S. Application Data
(63) Continuation-in-part of application No. 14/433,332, filed on Apr. 2, 2015.
(60) Provisional application No. 62/145,985, filed on Apr. 10, 2015.
(60) Provisional application No. 61/708,976, filed on Oct. 2, 2012.

(51) Int. Cl. A61N 7/02; G01S 7/52

(52) U.S. Cl. CPC A61N 7/02; G01S 7/52079; A61N 2007/0078

(57) ABSTRACT

This disclosure provides systems and methods for depositing a desired arbitrary plane and/or volume of ultrasound energy into a medium. This allows for programming of a desired arbitrary plane and/or volume of temperature elevation, computation of necessary operational parameters to generate the arbitrary plane and/or volume of temperature elevation, then rotation and translation of 1 to N ultrasound sources, optionally focused to different focal depths, with the calculated operational parameters.

### CROSS-REFERENCE TO RELATED APPLICATIONS

This application claims priority to and the benefit of U.S. Provisional Patent Application Ser. No. 61/708,976, entitled “Motion Mechanisms for Ultrasound Transducer Modules”, tiled Oct. 2, 2012, which is incorporated by reference herein.

### BACKGROUND

Focused ultrasound surgery has tremendous potential compared to other energy based treatment modalities. Ultrasound energy can be placed deep into tissue at precise depths with highly controlled spatial distributions. However, one difficulty has been treating a region or interest having a large volume. Attempts to address such problems have produced systems that are limited in flexibility and coverage to the scanned volume, not only of the treatment region but regions for imaging and monitoring. New systems and methods are needed for ultrasound treatment, which provide increased accuracy and flexibility of treatment and monitoring with ultrasound energy.

### SUMMARY

Various embodiments of systems and methods for ultrasound treatment provide motion mechanisms for use in ultrasound transducer modules. Various embodiments provide ultrasound treatment system comprising a transducer module and a control module.

In some embodiments, the transducer module can comprise a housing having a coupling surface connected to the upper surface by a contiguous wall and configured to retain a coupling solution, a movement mechanism located within the housing and comprising a rotatable shaft penetrating the upper surface of the housing, a first portion of an interface coupling on a distal end of the rotatable shaft and configured to mate with a second portion of the interface coupling, and at least one ultrasound transducer coupled to the movement mechanism and configured to emit ultrasound energy through the coupling surface and into a region of interest.

In some embodiments, the control module can comprise a drive device configured to controllably rotate the movement mechanism, and comprising the second portion of the interface coupling configured to mate with the first portion of the interface coupling, and a control system configured control a timing and a position of the ultrasound energy emitted by the at least one transducer.

In some embodiments, the transducer module is removeably attachable to the control module. In some embodiments, the first portion of the interface coupling is coupleable to the second portion of the interface coupling.

Further areas of applicability will become apparent from the description provided herein. It should be understood that the description and specific examples are intended for purposes of illustration only and are not intended to limit the scope of the embodiments disclosed herein.

### DRAWINGS

The drawings described herein are for illustrative purposes only and are not intended to limit the scope of any of the various embodiments disclosed herein or any equivalents thereof. It is understood that the drawings are not drawn to scale. Embodiments of the present disclosure will become more fully understood from the detailed description and the accompanying drawings, wherein:

FIG. 1 illustrates a treatment system, according to various embodiments of the present invention;

FIG. 2 illustrates an example of a treatment system, according to various embodiments of the present invention;

FIG. 3 illustrates a motion mechanism system, according to various embodiments of the present invention;

FIG. 4 illustrates a side cross-sectional view of an example configuration of a motion mechanism system, according to various embodiments of the present invention;

FIG. 5 illustrates a top cross-sectional view of an example configuration of a transducer module of FIG. 4, according to various embodiments of the present invention;

FIG. 6 illustrates a side cross-sectional view of another example configuration of a motion mechanism system, according to various embodiments of the present invention;

FIG. 7 illustrates a bottom view of an example configuration of a transducer assembly, according to various embodiments of the present invention;

FIG. 8 illustrates a expanded view of the partial cross-sectional view of an example configuration of a motion mechanism system comprising multiple transducers, according to various embodiments of the present invention;

FIG. 9 illustrates a side cross-sectional view of another example configuration of a motion mechanism system, according to various embodiments of the present invention;

FIG. 10 illustrates a side cross-sectional view of another example configuration of a motion mechanism system, according to various embodiments of the present invention;

FIG. 11 illustrates a perspective view of an example configuration of a transducer module comprising a housing, according to various embodiments of the present invention;

FIG. 12 is a chart illustrating different spans of imaging and treatment by a transducer, according to various embodiments of the present invention;

FIG.13 illustrates a side cross-sectional view of another example configuration of a motion mechanism system, according to various embodiments of the present invention;

FIG. 14 illustrates a perspective view of an example configuration of a transducer module comprising a housing, according to various embodiments of the present invention;

FIG. 15 illustrates a top view of a region of interest with an example of a treatment pattern, according to various embodiments of the present invention;

FIG. 16 illustrates a cross-sectional view of a region of interest with examples of treatment and imaging, according to various embodiments of the present invention; and

FIG. 17 illustrates a top view of a region of interest with an example of a circular pattern of treatment, according to various embodiments of the present invention.

### DESCRIPTION

The following description is merely exemplary in nature and is in no way intended to limit the various embodiments, their application, or uses. It should be understood that throughout the drawings, corresponding reference numerals indicate like or corresponding parts and features. The description of specific examples indicated in various embodiments of the present invention are intended for purposes of illustration only and are not intended to limit the scope of the invention disclosed herein. Moreover, recitation of multiple embodiments having stated features is not intended to exclude other embodiments having additional features or other embodiments incorporating different combinations of the stated features. Further, features in some embodiments (such as in one figure) may be combined with descriptions (and figures) of other embodiments.

The various embodiments may be described herein in terms of various functional components and processing steps. It should be appreciated that such components and steps may be realized by any number of hardware components configured to perform the specified functions. In addition, the embodiments may be practiced in any number of medical contexts and that the various embodiments relating to a method and system fur acoustic tissue treatment as described herein are merely indicative of exemplary applications for the invention. For example, the principles, features and methods discussed may be applied to any medical application. Further, various aspects of the various embodiments may be suitably applied to cosmetic applications. Moreover, some of the embodiments may be applied to cosmetic enhancement of skin and/or various subcutaneous tissue layers.

Various embodiments provide ultrasound treatment system comprising a transducer module and a control module.

In some embodiments, the transducer module can comprise a housing having a coupling surface connected to the upper surface by a contiguous wall and configured to retain a coupling solution, a movement mechanism located within the housing and comprising a rotatable shaft penetrating the upper surface of the housing, a first portion of an interface coupling on a distal end of the rotatable shaft and configured to mate with a second portion of the interface coupling, and at least one ultrasound transducer coupled to the movement mechanism and configured to emit ultrasound energy through the coupling surface and into a region of interest.

In some embodiments, the control module can comprise a housing configured to be hand-held. In some embodiments, control module can comprise a control system configured receive a position of transducer module and/or a speed of movement of the at least one transducer relative to the region of interest, and control a timing and a position of the ultrasound energy emitted by the at least on transducer based on the position and/or the speed.

In some embodiments, the control module can comprise a drive device configured to controllably rotate the movement mechanism, and comprising the second portion of the interface coupling configured to mate with the first portion of the interface coupling, and a control system configured control a timing and a position of the ultrasound energy emitted by the at least one transducer.

In some embodiments, the transducer module is removeably attachable to the control module. In some embodiments, the first portion of the interface coupling is coupleable to the second portion of the interface coupling.

In some embodiments, treatment system can comprise a position sensor configured to determine the position of the transducer module relative to the region or interest and in communication with the control module. In some embodiments, treatment system a motion sensor configured to determine speed of movement of the at least one transducer relative to the region of interest and in communication in communication with the control module.

The at least one transducer can comprises an array of transduction elements configured to vary a focal depth of the ultrasound energy and the control module configured to control the focal depth of the ultrasound energy. The at least one ultrasound transducer can comprise a plurality of ultrasound transducers, each coupled to the movement mechanism and controlled by the control module. In some embodiments, the at least one of the plurality of ultrasound transducers is configured to image at least a portion of the region of interest. The plurality of ultrasound transducers can be configured to have different focal depths of the ultrasound energy. The plurality of ultrasound transducers can be configured to have about the same focal depth of the ultrasound energy.

In some embodiments, the treatment system can comprise a comprising a graphic interface which can be in communication with the control module. In some embodiments, the transducer module can comprise a bubble entrapment system configured to trap coupling solution bubbles in a location away from a path of the ultrasound energy.

In some embodiments, the system can comprise a second energy source configured to direct a second energy into the region of interest comprising tissue. The second energy source can be one of a laser, intense pulse light, a light emitting diode, and a radiofrequency generator.

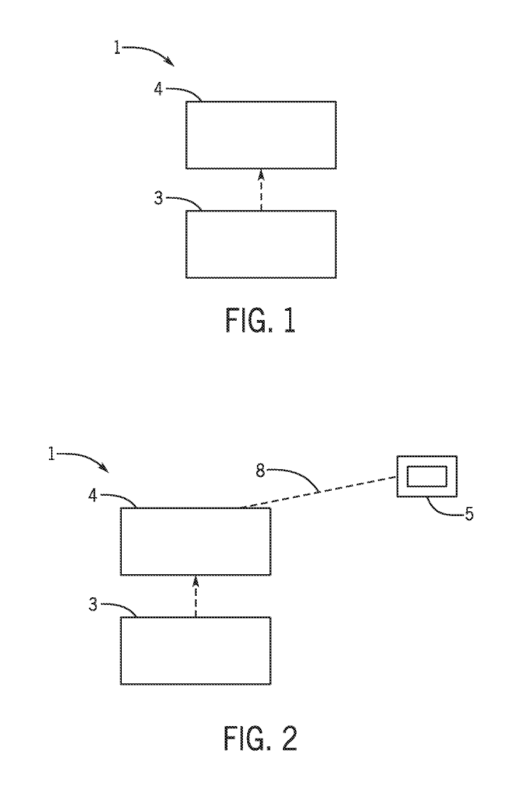

Referring to FIG 1, a treatment system 1 can comprise a control module 4 and a transducer module 3. Transducer module 3 can be self-contained and can be removably attached to control module 4. In various embodiments, transducer module 3 comprises at least one ultrasound transducer and at least a portion of a motion mechanism system. Transducer module 3 can comprise an acoustic coupling solution, which can be sealed within a housing of transducer module 3. Transducer module 3 can comprise a coupling surface configured to acoustically couple a transducer within transducer module 3 to a region of interest of a subject, such as, for example, subcutaneous tissue of a patient. One or more of the transducer can be configured to focus a conformal distribution of ultrasound energy into region of interest comprising subcutaneous tissue. One or more of the transducer can be configured to provide treatment into region of interest comprising subcutaneous tissue. One or more of the transducers can be configured to image the region of interest comprising subcutaneous tissue. The transducer, in any embodiment described herein, can be a therapy transducer, an imaging transducer, or an imaging and therapy transducer. The transducer in any embodiment described herein, can be a single element transducer or an array of transduction elements.

In various embodiments, one or more sensors may be included in the transducer module 3. For example, a sensor can be a magnetic sensor, such as a giant magnetoresistive effect (GMR) or Hall Effect sensor, and the encoder a magnet, collection of magnets, or multi-pole magnetic strip. The sensor may be positioned as transducer module 3 with a home position of a mechanical movement system. In some embodiments, a sensor can be a contact pressure sensor. For example, a sensor can be a contact pressure sensor on a coupling surface of transducer module 3 to sense the position of the treatment system 1 and/or transducer(s) on the patient. In various embodiments, a sensor can be used to map the position of treatment system 1 in one, two, or three dimensions. A sensor can be configured to sense the position, angle, tilt, orientation, placement, elevation, or other relationship between the treatment system 1 and/or transducer(s) and the patient. A sensor can be an optical sensor. A sensor can be a roller ball sensor. A sensor can be configured to map a position in one, two and/or three dimensions to compute a distance between areas or lines of treatment on the skin or subcutaneous tissue of a patient.

In some embodiments, the position sensor can be configured to communicate a position of the treatment system 1 and/or a speed of movement of treatment system 1. In some embodiments, the tissue contact sensor can be configured to communicate a coupling of the transducer to a region of interest of a subject.

Transducer module 3 can comprise a position sensor, which may be located behind transducer, in front of transducer, or integrated transducer. Transducer module 3 may comprise more than one position sensor, such as, for example, a laser position sensor and a motion sensor, or a laser position sensor and a visual device, or a motion sensor and a visual device, or a laser position sensor, a motion sensor, and a visual device. Additional embodiments of position sensor may be found in U.S. Pat. No. 7,142,905, entitled “Visual Imaging System for Ultrasonic Probe” issued Nov. 28, 2006, and U.S. Pat. No. 6,540,679, entitled “Visual Imaging System for Ultrasonic Probe” issued Apr. 1, 2003, both of which are incorporated by reference.

In some embodiments, transducer module 3 can comprise a communication interface, which communicates with the ultrasound transducer, motion mechanism, position sensor, and tissue contact sensor. In some embodiments, the communication interface communicates with ultrasound transducer, a position sensor, and a tissue contact sensor.

In some embodiments, a coupling solution can be sealed within transducer module 3.

In some embodiments, transducer module 3 has a bubble entrapment system, which can be configured to trap bubbles in the coupling solution in a location away from a path of the ultrasound energy emitted from transducer(s).

Control module 4 can comprise any combination of controllers, electronics graphic interface systems, and the like, which can control movement of and acoustic emission from transducer 10. Control module 4 can comprise a motor and be configured to control the motor, which can be coupled to a motion mechanism within transducer module 3. Control module 4 cart be configured to control a frequency of ultrasound sound energy emitted by each of the transducers in the transducer module 3. Control module 4 can be configured to control a power to transducer for ultrasound sound energy emitted by each of the transducers in the transducer module 3. Control module 4 can be configured to control the timing of ultrasound sound energy emitted by each of the transducers in the transducer module 3. Control module 4 can be located with a housing, which can be in a hand-held format. Control module 4 can be configured with an indicator, such as, for example, one or LED devices, to communication treatment system 1 conditions to a user. Control module 4 can be configured with safety warnings, such as, for example, audio alarms, flashing visual indicators, and/or system overrides. Control Module 4 can be configured to allow user to use treatment system 1 for limit allotted amounts of time.

Moving to FIG. 2, treatment system can comprise control module 4, transducer module 3, and a graphic interface 5. Control module 4 is in communication with graphic interface 5 via connection 8. In some embodiments, connection 8 is a wireless connection, such as, for example, a LAN, a WAN, Bluetooth, and the like, or any other wireless communication protocol now known or created in the future. Connection 8 can be coupled between control module 4 and graphic interface 5. In some embodiments, connection is a combination of both a wireless connection and a physical coupling between control module 4 and graphic interface 5.

Graphic interface 5 can display images generated by treatment system 1, for example, ultrasound images generated by use of transducer module 3. Graphic interface 5 can provide a user interface to control treatment system 1. Graphic interface 5 can comprise a portion of control protocol for controlling treatment system 1. Graphic interface 5 can comprise software to program and operate control module 4. Graphic interface 5 can collect data generated during treatment and/or monitoring of a subject. Graphic interface 5 can monitor transducer module 3 and terminate its use after a prescribed number of treatments. Graphic interface can provide safety protocol as required by various agencies, such as, for example the US Food and Drug Administration. Graphic interface can be any device, which can communication with the control module 4 and provide a display, such as, for example, a computer, a tablet, a iPad, a smartphone, or any other such device, now known or created in the future.

In some embodiments, control module 4 comprises at least one switch or button configured to control an emission of ultrasound energy from transducer module 3. In such embodiments, graphic interface 5 comprises various control protocol that is operable to control both transducer module 3 and control module 4. Transducer module can be configured to both image and treat subcutaneous tissue of a subject. Graphic interface can be configured to both control parameters of the treatment energy directed to a region of interest and to display an image of the region of interest.

Treatment system 1 can comprise a motion mechanism, which is coupled to the transducer(s) and is configured to be controlled by control module 4. In some embodiments, a slide coupled to transducer is driven by a motion mechanism, which may be located in control module 4 or transducer module 3. The motion mechanism can include a scotch yoke with a magnetic coupling configured to move the transducer. The motion mechanism can provide a more efficient, accurate and precise use of an ultrasound transducer, for imaging and/or for treatment purposes. One advantage this type of motion mechanism has over conventional fixed arrays of multiple transducers fixed in space in housing is that the fixed arrays are a fixed distance apart. By placing transducer on a linear rail and movement controlled by control module 4, embodiments of the system and device provide for adaptability and. flexibility in addition to the previously mentioned efficiency, accuracy and precision. Real time and near real time adjustments can be made to imagine and treatment positioning along the controlled motion by the motion mechanism. In addition to the ability to select nearly any resolution based on the incremental adjustments made possible by the motion mechanism, adjustments can be made if imaging detects abnormalities or conditions meriting a change in treatment spacing and targeting.

Motion mechanism can be any motion mechanism that may be found to be useful for movement of the transducer(s). Some embodiments of motion mechanisms useful herein can include worm gears and the like. In various embodiments of the present invention, at least a portion of the motion mechanism is located in the transducer module 3. Motion mechanism can provide for linear, rotational, multi-dimensional motion or actuation, and the motion can include any collection of points and/or orientations in space. Various embodiments for motion can be used in accordance with several embodiments, including but not limited to rectilinear, circular, elliptical, arc-like, spiral, a collection of one or more points in space, or any other 1-D, 2-D, or 3D positional and attitudinal motional embodiments. The speed of the motion mechanism may be fixed or may be adjustably controlled by a user. Some embodiments, a speed of the motion mechanism for an image sequence may be different than that for a treatment sequence. In some embodiments, the speed of the motion mechanism is controllable by control module 4. A motion mechanism with feedback can be controlled to scan a transducer array in a linear pattern or a two-dimensional pattern or over a varied depth, which can be varied by adjusting parameters of the transducer array.

In some embodiments, motion mechanism can be configured to scan ultrasound transducer(s) in at least one of a linear pattern, a two-dimensional pattern and a three-dimensional pattern, and provide cosmetic enhancement to a patient. The term “cosmetic enhancement” can refer to procedures, which may not be medically necessary but can be used to improve or change the appearance of a portion of the body. For example, a cosmetic enhancement can be a procedure but not limited to procedures that are used to improve or change the appearance of a nose, eyes, eyebrows and/or other facial features, or to improve or change the appearance and/or the texture and/or the elasticity of skin, or to improve or change the appearance of a mark or scar on a skin surface, or to improve or change the appearance and/or the content of fat near a skin surface, or the targeting of a gland to improve or change the appearance a portion of the body. As used herein “cosmetic enhancement” can be a non-surgical and non-invasive procedure. In various embodiments, cosmetic enhancement provides rejuvenation to at least one portion of the body. Rejuvenation can be the reversal of aging and is namely repair of the damage that is associated with aging or replacement of damaged tissue with new tissue.

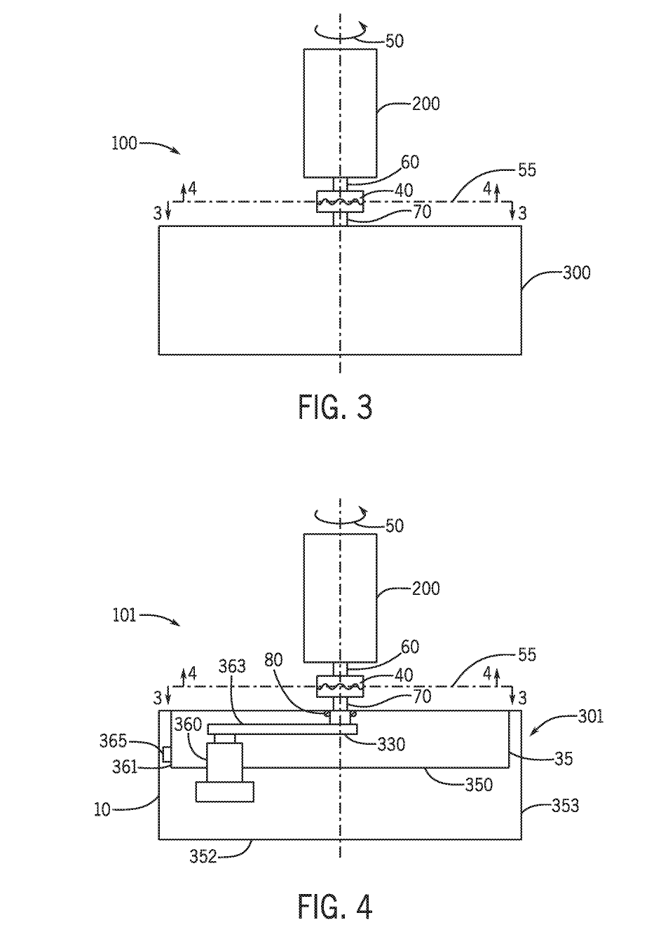

With reference to FIG. 3, a motion mechanism system is illustrated. In various embodiments, a motion mechanism system 100 comprises a motor 200 providing rotational motion into an example configuration of transducer module 3, mainly transducer module 300. Motion mechanism system 100 can include a motor shaft 60 directing rotational energy to an interface coupling 40 and to a module shalt 70, which is engaged into transducer module 300. Interface coupling 40 is in two parts which couple upon attachment of transducer module 3 to control module 4. Interface coupling made be keyed to couple in a home position, which can be communicated to control module 4.

Motion Mechanism 100 is coupled to ultrasound transducer 10 and can be configured to scan the ultrasound transducer 10 in at least one of a linear pattern and a two-dimensional pattern. Motion mechanism 100 is coupled to ultrasound transducer 10 and can be configured to scan the ultrasound transducer 10 to provide treatment in a two-dimensional pattern in subcutaneous tissue of a patient

Motion mechanism system 100 can be divided into two portions as illustrated by line 55 which bisects interface coupling 40, which can be, for example, a magnetic interface. Control module 4 comprises a portion of motion mechanism 100, which is above line 55. Transducer module 3 comprises a portion of motion mechanism 100, which is below line 55. Control Module 4 can comprise any combination of controllers, electronics graphic interface systems, and the like, which can control movement of and acoustic emission from transducer 10. Transducer module 3 can include any combination of transducers, sensors, motion sensors, coupling solutions, coupling fluids, acoustic windows, matching layers and the like.

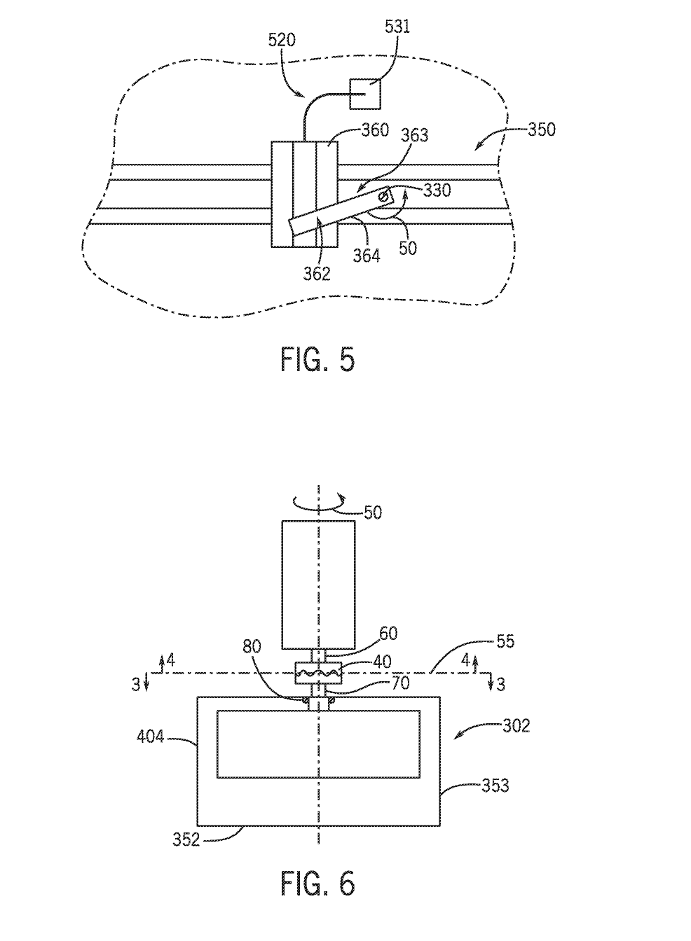

In some embodiments, motion mechanism system 101 comprises an example configuration of transducer module 3, mainly transducer module 301, as illustrated in FIGS. 4 and 5. Transducer module 301 can comprise a transducer 10 mounted to a slide 360 which is interfaced to a tail 350. Transducer module 301 can comprise a housing 353, which may comprise an acoustic window 352 and an opening. A seal 80, which is configured to seal the opening, can be engaged with a transducer shaft 70, which is positioned in the opening. Slide 360 can be configured for movement of transducer 10 parallel to rail 350 from a stop 361 at one end of rail 350 to a distal stop 369 at the other end of rail 350.

Rotational movement (indicated by arrow 50) can spin arm 363, which is engaged with, slide 360. Arm 363 rotates around a pivot point 330, which can be at the end of the transducer shaft 70. Movement of slide 360 linearly along rail 350 is generated by movement of arm 363 around pivot point 330. Movement of arm 363 is controlled by a speed and direction of rotation of transducer shaft 70. Arm 363 can be configured to be engaged in slot 362, such that rotational motion 50 allows slide 360 to move in either direction along rails 350. In some embodiments, an electronics interface 520 can be coupled to transducer 10, for interfacing transducer 10 with system.

Some embodiments of motion mechanism system can comprise a two phase stepper motor and a scotch yoke to produce rotational motion. The stepper motor which can moves a pin in a circular path. The pin slides in a slot of the slide. This causes the slide to move in a linear fashion. The slide is held by rails and glide members may be between the slide and the rails. Embodiments of the glide member may include any material or mechanical device that lowers a coefficient of friction between the slide and the rail, or any linear bearings. For example, in various embodiments the glide member can be at least one of an elastomeric material, a lubricant, ball bearings, a polished surface, a magnetic device, pressurized gas, or any other material or device useful for gliding. In some embodiments, the transduction element of the transducer is scanned in a linear motion to cover the region of interest, such that at any time the energy is not coming out of the entire transducer housing's length at once.

In some embodiments, electronics interface 520 can be coupled to transducer 10. Electronics interface 520 can provide power to transducer 10. Electronic interface 520 can deliver communication from control module 4. Electronic interface 520 can send data from transducer 10 to control module 4. In some embodiments, a portion of electronic interface 520 is coupled to a sensor in or on transducer module 3. Electronic interface 520 can provide a feedback loop between control module 4 and sensor. In some embodiments, sensor can be a position sensor. In some embodiments, sensor can be a contact sensor. In some embodiments, sensor can be a temperature sensor. Electronic interface 520 can be fitted through port 521. Electronic interface 520 can be coupled to port 521. A housing enclosing transducer module 3 can comprise port 521. Port 521 is configured to provide a seal to or around electronic interface 520 and prevent leaking of coupling solution from transducer module 3. Port 521 can comprise an antenna configured for communication with control module 4 and/or graphic interface 5 over a wireless network.

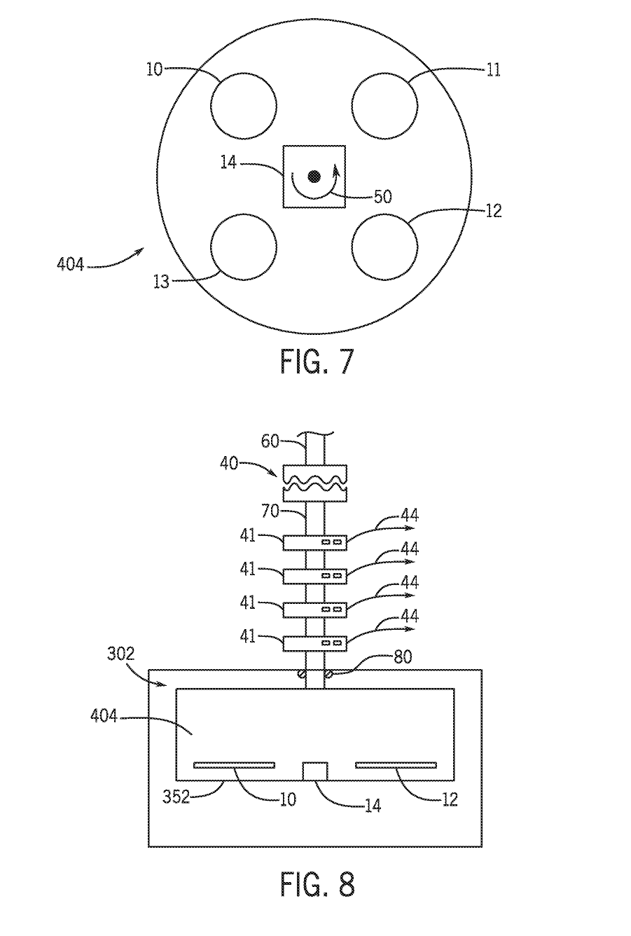

In some embodiments, motion mechanism 102 comprises an example of transducer module 3, mainly transducer module 302, as illustrated in FIGS. 6-8. Transducer module 302 comprises transducer assembly 404 which can have a rotational movement (indicated by arrow 50). Transducer assembly 404 can comprise a plurality of transducers. In an example, transducer assembly 404 comprises first transducer 10, a second transducer 11, of three transducer 12, and a fourth transducer 13. In another example transducer assembly 404 can comprise imaging transducer 14. Imaging transducer 14 can located on acoustic window 352, which can improve an image produced of region of interest. The transducers, in this embodiment, can be any of a therapy transducer, an imaging transducer, or an imaging and therapy transducer. Any of the transducers, in this embodiment, can be a single element transducer or an array of transduction elements.

With reference to FIG. 8, expanded view of the partial section of motion mechanism system 102 is illustrated. Transducer assembly 404 can comprise multiple electronic interface 44, which provides a means of control and/or power directed from treatment system to transducer located in transducer assembly 404. In some embodiments, transducer shaft 70 is hollow and comprises a plurality of slip rings 41, which can have an inner portion that can spin with shaft 70 and an otter portion that can spin independent of shaft 70. Electronic interface 44 can be coupled to slip ring 41, such that transducer shaft 70 can spin without damaging, breaking, or tangling electronic interface 44. Slip ring 41 can be configured to provide inductive coupling between electronic interface 44 and coupling to a transducer. A coupling to a transducer can be connected to an internal portion of slip ring 41, which positioned within hollow core of transducer shaft 70 and can spin with transducer shaft 70. Internal portion of slip ring 41 may be part of inner portion of slip ring 41. A coupling to a transducer can be etched onto inner wall of transducer shaft 70. A coupling to a transducer can be laminated onto inner wall of transducer shaft 70.

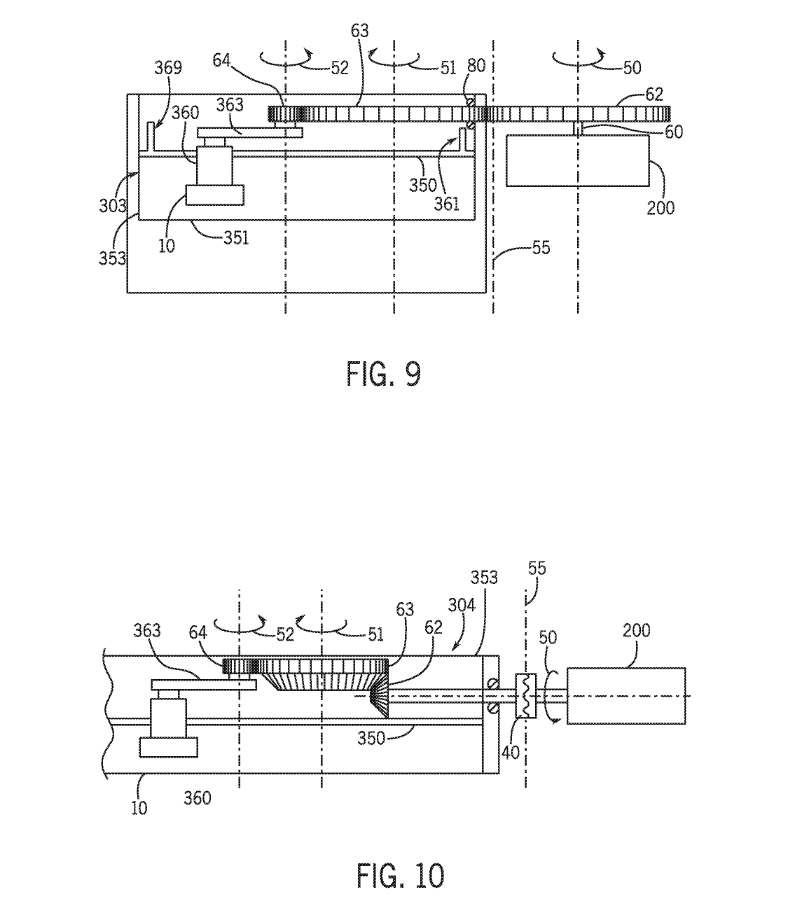

In some embodiments, motion mechanism 103 can comprise a plurality of gears, as illustrated in FIG. 9. In some embodiments motor 200 comprises motor shaft 60 which is coupled to first gear 62 having a rotational motion 50. As will be apparent to one skilled in the art, rotational motion is illustrated by 50 could be in either direction that is clockwise or counterclockwise in this or any other embodiment described herein. First gear 62 can be interfaced to second gear 63 allowing motion to be transferred from motor 200 to transducer module 303. Second gear 63 has rotational motion 51, which typically is opposite in direction of the motion 50 of first gear 62. Second gear 63 transfers motion to third gear 64, which has rotational motion 52. Arm 363 pivots around pivot point 330, which provides slide 360 and transducer 10 movement, which is similar to that illustrated in FIG. 5,

In some embodiments, motion mechanism system 104 can comprise a plurality of gears, as illustrated in FIG. 10. In some embodiments motor 200 comprises motor shaft 60 which is coupled to first gear 62 having a rotational motion 50. First gear can be interfaced to second gear 63 allowing motion to be transferred from motor 200 to transducer module 303. Second gear 63 has rotational motion 51, which typically is opposite in direction of the motion 50 of first gear 62. Second gear 63 transfers motion to third gear 64, which has rotational motion is illustrated by 52. 30 or 64 pivots around pivot point 33, which provides slide 360 and transducer 10 movement, which is similar to that illustrated in FIG. 5.

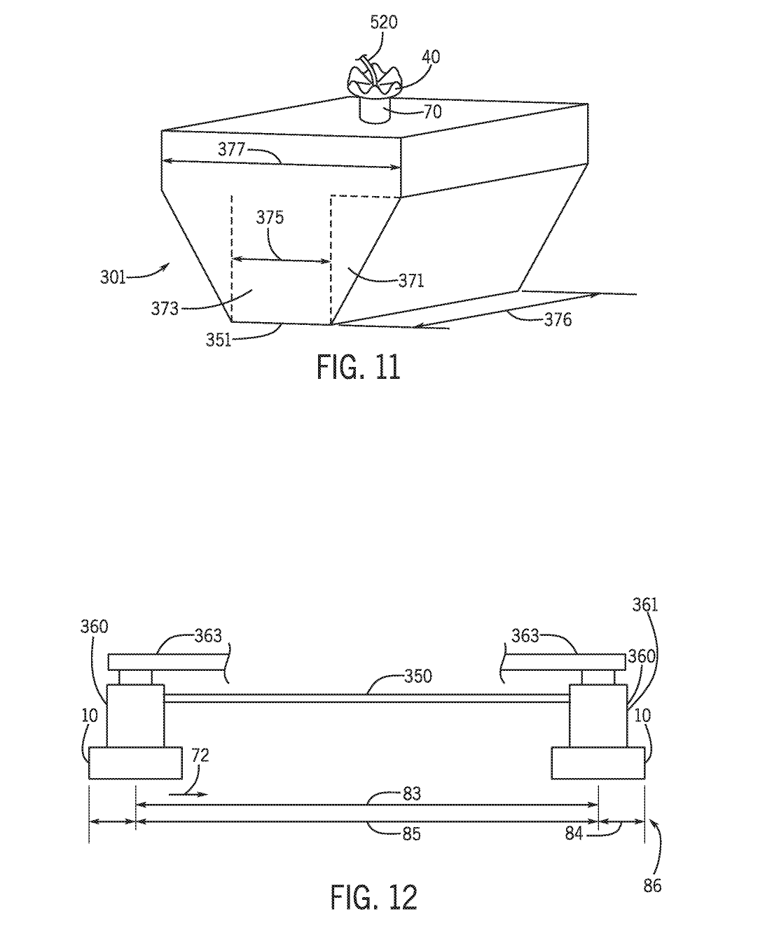

As illustrated in FIG. 11, a non-limning example of transducer module comprising housing 353 is provided. In some embodiments, illustrated herein, housing 353 has acoustic window 351 on bottom location which has a span 375 equivalent to the width of energy provided by a transducer. Rotational span 377 is equivalent to at least the diameter of the spin of arm 363 plus allowance for housing thickness and tolerance. As will be apparent to one skill in the art, acoustic rotational span 377 can be greater than that described. Housing length 376 is equivalent to at least the travel of the transducer within housing 353.

As illustrated in FIG. 12, this span can vary, for example, the span of imaging and/or treatment may be from a center point of a stop of transducer 10 to center point of opposite stop, as illustrated by the prime, which is shown as effective span 83. However the actual travel is effective span 83 plus at least half of transducer 10 which is illustrated by travel span 86. In some embodiments length 376 is no less than travel span 86 plus tolerance and thickness for housing. In some embodiments as illustrated by area 371, void, which is tapered towards energy span 373 in area 371, can comprise mechanisms for cooling for the transducer and/or the tissue in a region of interest targeted by the transducer.

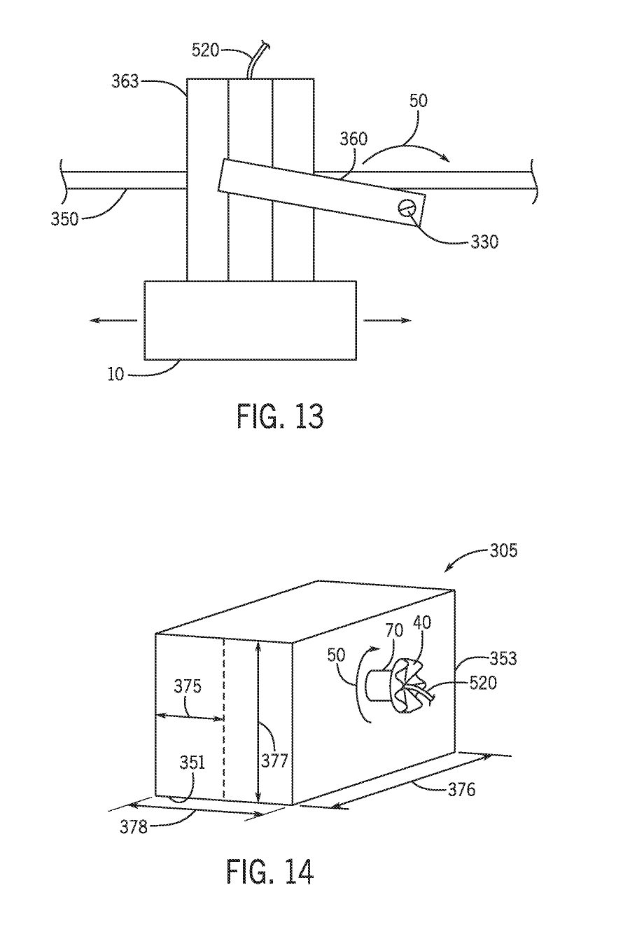

In some embodiments rotation direction can be transferred in a plane that is parallel to rotation 50. As illustrated in FIG. 13, this mechanism can be transferred to any of the embodiments and examples described herein. As Shown in FIG. 14, housing 353 is illustrated for the mechanism, as described in FIG. 13. In this example rotational span 377 is in the same plane however direction energy span 375 is in a different plane, as shown a distance of thickness 378 is equal to the mechanical portion allowing rotation plus energy span 375.

A variety of embodiments of creating lesions patterns are described herein. However, in various embodiments, transducer module 3 comprises position sensor. Position sensor can be integrated into transducer module 3 or attached to transducer module 3. In some embodiments, position sensor is a motion sensor measuring position of ultrasound probe. Such a motion sensor can calculate distance traveled along skin surface. Such a motion sensor may determine a speed of movement of transducer module along skin surface and determine if the speed is accurate for treatment. For example if the speed is too fast, motion sensor can signal an indicator to slow the speed and/or can signal therapy transducer to stop emitting therapeutic ultrasound energy. In some embodiments, a position sensor can be located on a rotating surface of transducer module 3 and position can be calculated as polar coordinates.

In some embodiment, transducer module 3 can be configured to direct ultrasound energy area of treatment in a range from approximately 100 microns to 55 mm in diameter. In some embodiments, ultrasound energy can be configured in a “lawnmower” type fashion to evenly ablate a treatment region to provide a substantially planar surface at a depth in subcutaneous tissue, This “lawnmower”-type ablation can achieve a substantially smooth surface of skin above the planar surface at a depth in subcutaneous tissue. In some embodiments, transducer module 3 can be configured to deliver ultrasound energy as “carpet bomb” of subcutaneous tissue at 1-7 mm depth.

In some embodiments, transducer module 3 can be configured creating a three dimensional matrix of lesions in subcutaneous tissue at one depth or at a multitude of depths.

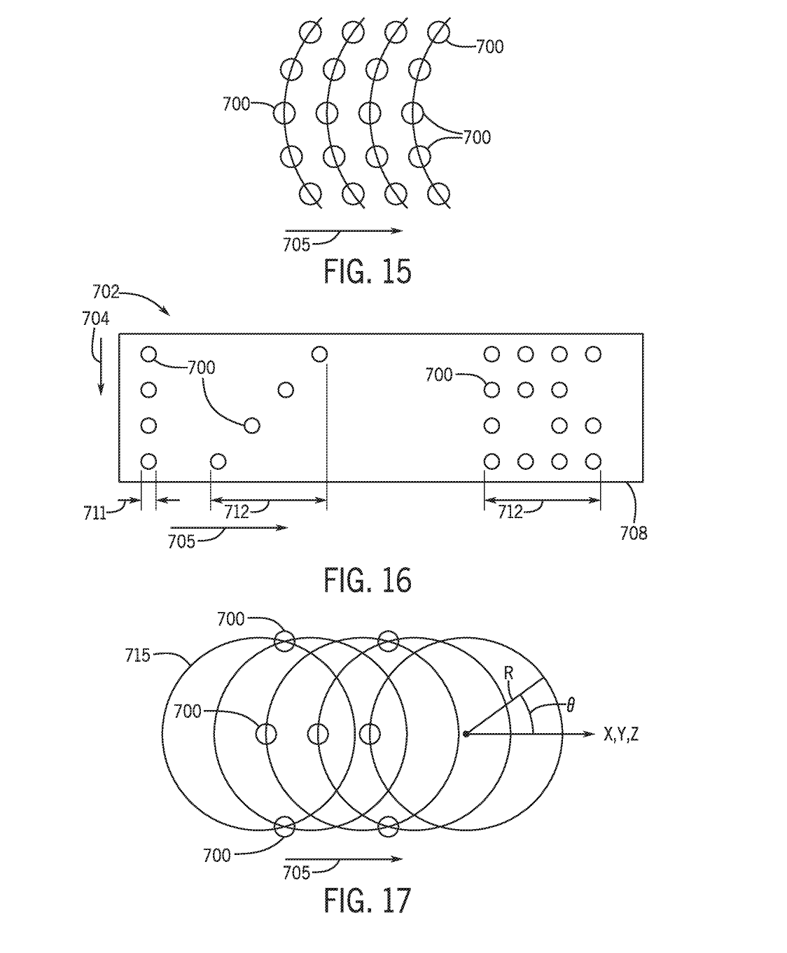

In one non-limiting example, FIG. 15 illustrates a top view of a region of interest with an example of a treatment pattern, according to various embodiments of the present invention. As treatment system is moved in direction 705, transducers can create an array of lesions 700 along an arc 707. A depth of each of the lesion 700 can be about equivalent to each other. However, a depth of each lesion 700 long arc 707 can at different depths.

In a non-limiting example FIG. 16 illustrates a cross-sectional view of a region of interest with examples of treatment and imaging, according to various embodiments of the present invention. A region of interest 702 can be imaged in filed as illustrated by box 708. A portion of the region of interest 702 can have a plurality of lesions 700 at a plurality of depth 704 as illustrated in section 711. A portion of the region of interest 702 can have a plurality of lesions 700 at a plurality of depth 704 and a plurality of distance, as illustrated in section 712. A portion of the region of interest 702 can have a plurality of lesions 700 at a variety of depths 704 and a variety of distances, as illustrated in section 712. Treatment system 1 can be configured to produce a plurality of lesions in a three dimensional volume.

In a non-limiting example, FIG. 17 illustrates a top view of a region of interest with an example of a circular pattern of treatment, according to various embodiments of the present invention. Circular path 725 can be produce by, for example, a treatment system illustrated in FIGS. 6-8. As system moves along path 705, circular path 715 can produce a matrix of evenly spaced lesions 700. In some embodiments, treatment system can use polar coordinates to produce any configuration of lesions 700 in a treatment volume.

Various embodiments provide treatment system 1 for treating soft tissue. In some embodiments, treatment systems can include transducer module 3, and control module 4 comprising a power supply. In some embodiments, treatment systems can include transducer module 3, control module 4 comprising a power supply, and graphic interface 5 operable to communicate with control module 4. In some embodments, transducer module 3 can comprise an ultrasound transducer, a motion mechanism, a position sensor, a tissue contact sensor, and a communication interface. Control module 4 can communicates with the communication interface and can be configured to control a spatial parameter and a temporal parameter of the ultrasound transducer to emit the conformal distribution of ultrasound energy into a region of interest of a subject. In some embodiments, the ultrasound transducer configured to focus a conformal distribution of ultrasound energy into a region of interest comprising tissue. In some embodiments, the ultrasound transducer is configured to image the region of interest comprising tissue.

Power supply can be rechargeable and can comprise one or more batteries. Power supply can supply power to transducer module comprising the ultrasound transducer, and at least a portion of the motion mechanism. Various embodiments provide treatment system 1 in a hand held format comprising control module 4, which further comprises a rechargeable power supply, and a transducer module 3, which further comprises a dual-function ultrasound transducer, a motion mechanism, a position sensor, and a communication interface. In some embodiments, the dual-function ultrasound transducer can be configured to focus a conformal distribution of ultrasound energy into a region of interest comprising tissue.

In some embodiments, the communication interface can be configured for wireless communication. Communication interface can communicate with transducer module 3 comprising the dual-function ultrasound transducer, the motion mechanism, and the position sensor. Rechargeable power supply can supply power to the dual function ultrasound transducer, the motion mechanism, the position sensor, and the communication interface.

Control module 4 can communicate with the communication interface. In some embodiments, at least one of control module 4 and graphic interface 5, or a combination thereof, controls a spatial parameter and a temporal parameter of the dual-function ultrasound transducer to emit the conformal distribution of ultrasound energy. In some embodiments, control module 4 receives the position of treatment system 1 and the speed of movement of transducer(s) in transducer module3. In some embodiments, at least one of control module 4 and graphic interface 5, or a combination thereof, controls the timing of conformal distribution of ultrasound energy based on the position and the speed of transducer module 3. In some embodiments, at least one of control module 4 and graphic interface 5, or a combination thereof, controls the scan of transducer(s) with the motion mechanism based on the position and the speed.

In some embodiments, the controller is configured to receive the at least one of the position of the housing and the speed of movement of the housing, and is configured to control the timing of conformal distribution of ultrasound energy based on at least one of the position and the speed. In some embodiments, the controller is configured to receive the at least one of the position of the housing and the speed of movement of the housing, and is configured to control the scan of the motion mechanism based on at least one of the position and the speed.

Speed of motion can be used to control therapeutic ultrasound energy. For example, if the motion is too fast information can be provided to the user to slow down and/or energy can be dynamically adjusted within limits. Position information may also be used to suppress energy if crossing over the same spatial position, if desired. Such a position sensor may also determine if transducer module 3 is coupled to skin surface, to safely control energy delivery and provide information to users. Position sensor data acquisition can be synchronized with imaging sequence and monitoring sequence, to geo-tag and arrange the image frames in the correct spatial orientation to form an extended image, or likewise extended monitoring image, for display.

In some embodiments, control module 4 can receive the position of transducer module 3 and/or the speed of movement of the transducer relative to region of interest, and can control the timing of conformal distribution of ultrasound energy based on the position and/or the speed. In some embodiments, control module 4 can receive the position of the transducer module and/or the speed of movement of the transducer module and can control the scan of the motion mechanism based on the position and/or the speed.

In some embodiments, treatment system 1 can include a second energy source configured to direct a second energy into the region of interest comprising tissue. A second energy source may be one of a laser, intense pulse light, a light emitting diode, and a radiofrequency generator. A second energy source can be a combination of at least two energy sources, such as for example combination of at least two of a laser, an intense pulsed light, a light emitting diode, a radiofrequency generator, an acoustic source, and/or a mechanical energy source, can be instantaneous and emitted simultaneously or with a time delay. In various embodiments, energy may be a first energy and a second energy. For example, a first energy may be followed by a second energy, either immediately or after a delay period. In another example, a first energy and a second energy can be delivered simultaneously. In some embodiments, the first energy and the second energy is ultrasound energy. In some embodiments, the first energy is ultrasound and the second energy is generated by one of a laser, an intense pulsed light, a light emitting diode, a radiofrequency generator, or a mechanical energy source, such as for example, pressure, either positive or negative. In other embodiments, energy may be a first energy, a second energy, and a third energy, emitted simultaneously or with a time delay or a combination thereof. In some embodiments, energy may be a first energy, a second energy, a third energy, and an nth energy, emitted simultaneously or with a time delay or a combination thereof. Any of the a first energy, a second energy, a third energy, and a nth energy be generated by at least one of a laser, an intense pulsed light, a light emitting diode, a radiofrequency generator, an acoustic source, and/or a mechanical energy source.

Treatment system 1 as described herein, as a primary source of treatment, may be combined with a second treatment function configured to deliver a second treatment energy. Second treatment energy can include, but is not limited to, any of radio frequency (RF) energy, microwave energy, infrared light, visible light, ultraviolet light, or any other suitable electromagnetic energy. Second treatment energy may be coherent (as in a laser), incoherent, scattered, pulsed, refracted, focused, defocused, and/or delivered in any other form suitable for achieving a bio-effect.

In an exemplary embodiment, ultrasound treatment is combined with blue light treatment. As used herein, “blue light” means electromagnetic energy having a wavelength from about 400 nanometers to about 440 nanometers. Blue light is applied to the skin. Blue light may be applied as a pretreatment before therapeutic ultrasound energy is applied. Blue light may also be applied concurrently with therapeutic ultrasound energy. Furthermore, blue light may be applied before, during, or after therapeutic ultrasound treatment, or during any combination thereof.

In accordance with an exemplary embodiment, blue light is applied to treatment zone 100 for a period between 5 seconds and 20 minutes. Blue light may be applied to treatment zone 100 for any suitable amount of time in order to achieve a desired bio-effect.

In another exemplary embodiment, ultrasound treatment is combined with red light treatment. As used herein, “red light” means electromagnetic energy having a wavelength from about 600 nanometers to about 1350 nanometers. Red light is applied to treatment zone 100. Red light may be applied as a pretreatment before therapeutic ultrasound energy is applied. Red light may also be applied concurrently with therapeutic ultrasound energy. Furthermore, red light may be applied before, during, or after therapeutic ultrasound treatment, or during any combination thereof.

In accordance with an exemplary embodiment, red light is applied to the skin for a period between 5 seconds and 20 minutes. Red light may be applied to the skin for any suitable amount of time in order to achieve a desired bio-effect.

In accordance with an exemplary embodiment, secondary treatment energy can be delivered by the probe which contains an ultrasound energy source. In other exemplary embodiments, secondary treatment energy is delivered by a source external to the probe. Secondary treatment energy may be generated by a light emitting diode (LED), a laser, an incandescent bulb, a fluorescent tube, an antenna, an intense pulsed light source, or any other suitable electromagnetic energy generation mechanism,

In some embodiments, transducer module 3 is configured to removably attach both electronically and mechanically to control module 4. In some embodiments, a motion mechanism is configured to move ultrasound transducer(s) in transducer module 3 such as is illustrated in various embodiments described herein. A coupling liquid can be sealed within transducer module 3. In some embodiments, transducer module 3 has a bubble entrapment system, which can be configured to trap bubbles in the coupling liquid in a location away from a path of the ultrasound energy emitted from transducer(s). User can remove a transducer module 3 from its protective, resealable pouch, setting aside the pouch for storing the transducer module 3 between procedures, if necessary. In some embodiments, transducer module 3 can be connected to control module 4 by pushing transducer module 3 into control module 4. In some embodiments, when the transducer module 3 is inserted, control module 4 automatically detects it and updates the interactive graphical display. In some embodiments, transducer module 3 is locked into control module 4 once the transducer module 3 is fully inserted and a coupling mechanism has been engaged.

In any of the embodiments disclosed herein, imaging occurs prior to the therapy, simultaneously with the therapy, or after the therapy. In several of the embodiments described herein, the procedure is entirely cosmetic and not a medical act.

The following patents and patent applications are incorporated by reference: US Patent Application Publication No, 20050256406, entitled “Method and System for Controlled Scanning, Imaging, and/or Therapy” published Nov. 17, 2005; US Patent Application Publication No. 20060058664, entitled “System and Method for Variable Depth Ultrasound Treatment” published Mar. 16, 2006; US Patent Application Publication No. 20060084891, entitled Method and System for Ultra-High Frequency Ultrasound Treatment” published Apr. 20, 2006; U.S. Pat. No. 7,530,958, entitled “Method and System for Combined Ultrasound Treatment” issued May 12, 2009; US Patent Application Publication No. 2008071255, entitled “Method and System for Treating Muscle, Tendon, Ligament, and Cartilage Tissue” published Mar. 20, 2008; U.S. Pat. No. 6,623,430, entitled “Method and Apparatus for Safely Delivering Medicants to a Region of Tissue Using Imaging, Therapy, and Temperature Monitoring Ultrasonic System, issued Sep. 23, 2003; U.S. Pat. No. 7,571,336, entitled “Method and System for Enhancing Safety with Medical Peripheral Device by Monitoring if Host Computer is AC Powered” issued Aug. 4, 2009; and US Patent Application Publication No. 20080281255, entitled “Methods and Systems for Modulating Medicants Using Acoustic Energy” published Nov. 13, 2008; US Patent Application Publication No 20080281255, entitled “Methods and Systems for Modulating Medicants Using Acoustic Energy” published Nov. 13, 2008; US Patent Application Publication No. 20060116671, entitled “Method and System for Controlled Thermal Injury of Human Superficial Tissue,” published Jun. 1, 2006; US Patent Application Publication No. 20060111744, entitled “Method and System for Treatment of Sweat Glands,” published May 25, 2006; US Patent Application Publication No. 20080294073, entitled “Method and System for Non-Ablative Acne Treatment and Prevention,” published Oct. 8, 2009; U.S. Pat. No. 8,133,180, entitled “Method and System for Treating Cellulite,” issued Mar. 13, 2012; U.S. Pat. No. 8,066,641, entitled “Method and System for Photoaged Tissue,” issued Nov. 29, 2011; U.S. Pat. No. 7,491,171, entitled “Method and System for Treating Acne and Sebaceous Glands,” issued Feb. 17, 2009; U.S. Pat. No. 7,615,016, entitled. “Method and System for Treating Stretch Marks,” issued Nov. 10, 2009; U.S. Pat. No 7,530,356, entitled “Method and System for Noninvasive Mastopexy,” issued May 12, 2009; US Patent Applicant Publication No. 20100160782, entitled. “Methods and Systems for fat Reduction and/or Cellulite Treatment,” published Jun. 24, 2010; and US Patent Application Publication No. 20120046547, entitled “System and Method for Cosmetic Treatment,” published Feb. 23, 2012.

In the foregoing specification, the invention has been described with reference to specific embodiments. Various modifications and changes may be made, however, without departing from the scope of the various embodiments of the present invention, as set forth in the claims. The specification and Figures are illustrative, rather than restrictive, and modifications are intended to be included within the scope of any of the various embodiments of the present invention described herein. Accordingly, the scope of the invention should be determined by the claims and their legal equivalents rather than by merely the examples described.

For example, the steps recited in any method or process claims may be executed in any order and are not limited to the specific order presented in the claims. Additionally, the components and/or elements recited in any apparatus or system claims may be assembled or otherwise operationally configured in a variety of permutations and are accordingly not limited to the specific configuration recited in the claims.

Benefits, other advantages and solutions to problems have been described above with regard to particular embodiments; however, any benefit, advantage, solution to problem or any element that may cause any particular benefit, advantage or solution to occur or to become more pronounced are not to be construed as critical, required or essential features or components of any or all the claims.

### Disclosure

This disclosure provides a system and method for depositing a desired arbitrary plane of ultrasound energy into a medium. The system and method allow for programming of a desired arbitrary plane of temperature elevation, computation of the necessary operational parameters to generate that arbitrary plane of temperature elevation, then rotation and translation of 1 to N ultrasound sources with the calculated operational parameters.

This disclosure also provides a system and method for depositing a desired arbitrary volume of ultrasound energy into a medium. The system and method allow for programming of a desired arbitrary volume of temperature elevation, computation of the necessary operational parameters to generate that arbitrary volume of temperature elevation, then rotation and translation of a plurality of groups of 1 to N ultrasound sources focused to different focal depths with the calculated operational parameters.

### Arbitrary Plane

The systems and methods can comprise 1 to N ultrasound sources each having a focus at the depth at which the arbitrary plane is desired. The 1 to N ultrasound sources can be offset from a rotational axis. When the ultrasound 1 to N ultrasound sources are rotated about the rotational axis and translated linearly along a surface above the location where the arbitrary plane is desired, the ultrasound energy emitted from the 1 to N ultrasound sources is transmitted to the depth of the arbitrary plane where it creates an arbitrary variation in temperature in the same arbitrary plane.

For instance, a single ultrasound source offset from the rotational axis that emits continuous ultrasound will create a circle of elevated temperature parallel to the surface if rotated but not linearly translated. Linearly translating the rotating arrangement will translate the circle of elevated temperature in the linear direction, thereby creating the plane of elevated temperature along the path of the translating circle. If using two or more ultrasound sources, each source can be located at the same distance from the rotational axis (thereby producing a single circle when not moving linearly) or at different distances from the rotational axis (thereby producing concentric circles with not moving linearly).

As another instance, a single ultrasound source offset from the rotational axis that emits pulsed ultrasound that is synced with the rotation about the rotational axis will create discrete conformal distributions of ultrasound energy at points along a circle parallel to the surface if not linearly translated. Linearly translating this rotating arrangement will translate the points in the linear direction, thereby creating lines of elevated temperature in a plane at the focal depth of the

As another instance, a single ultrasound source offset from the rotational axis that emits pulsed ultrasound that is not synced with the rotation about the rotational axis will again create a circle of elevated temperature parallel to the surface if not linearly translated. Linearly translating this rotating arrangement will behave as the continuous example described above.

Emitting pulsed ultrasound at a constant repetition rate, the spacing between depositions can be varied by changing the rotational velocity and/or the linear velocity.

The density of coverage can be selectively controlled by varying the rotational velocity, the linear velocity, and the timing and intensity of the ultrasound deposition.

The relative geometry of the ultrasound sources can be dictated by the intensity gain between the ultrasound source and the desired arbitrary plane.

A mathematical computation can be performed in either polar coordinates or Cartesian coordinates to determine the necessary rotational velocity, the necessary linear velocity, and the necessary timing and intensity of ultrasound emission required to create the arbitrary plane of elevated temperature. A control module can be programmed with the desired arbitrary plane of elevated temperature, and the computation can calculate the necessary temporal and spatial parameters to produce that plane. In certain aspects, the computation can determine the number of ultrasound sources necessary to produce the arbitrary plane of elevated temperature. In certain aspects, the computation can determine the necessary temporal and spatial parameters for one ultrasound source, two ultrasound sources, three ultrasound sources, up to N ultrasound sources.

In order to change the treatment time, the number of ultrasound sources can be increased, which also allows the rotational velocity to be slowed.

Arbitrary Volume

The systems and methods can comprise 1 to N first ultrasound sources having a focus at a first depth, 1 to N second ultrasound sources having a focus at a second depth, and optionally 1 to N third, fourth, fifth, up to nth ultrasound sources having a focus at a third, fourth, fifth, up to nth depth, respectively.

Depositing an arbitrary volume operates on essentially the same set of principles as depositing an arbitrary plane, considering each different focal depth to be its own arbitrary plane and then adding the effect of the various focal depths to create the arbitrary volume.

A mathematical computation can be performed in either spherical coordinates or Cartesian coordinate to determine the necessary rotational velocity, the necessary linear velocity, and the necessary timing and intensity of ultrasound emission required to create the arbitrary volume of elevated temperature. A control module can be programmed with the desired arbitrary volume of elevated temperature, and the computation can calculate the necessary temporal and spatial parameter to produce that plane.

The ultrasound sources described herein can be any shape, round, not round, trapezoidal, etc.

The ultrasound effect generated in the arbitrary plane or volume can be any desired effect.

The arbitrary plane described herein can be at the surface of the medium. The arbitrary volume described herein can include the surface of the medium.

International Patent Application No. PCT/US2013/063150, filed Oct. 2, 2013, and U.S. Provisional Patent Application No. 61/708,976, filed Oct. 2, 2012, are incorporated herein in their entirety by reference.

1. A method of generating an arbitrary plane of elevated temperature in a medium at a surface of the medium or at a depth beneath the surface of the medium, the method comprising:
calculating the necessary operational parameters to generate the arbitrary plane of elevated temperature;
rotating one or more ultrasound sources about a rotational axis and linearly translating the one or more ultrasound sources while emitting ultrasound energy from the ultrasound sources,
each ultrasound source having a focal depth at the surface or at the depth beneath the surface,
the rotating, the linearly translating, and the emitting being executed according to the calculated operational parameters.

2. A method of generating an arbitrary volume of elevated temperature in a medium at a range of depths, the range of depths selected from a surface of the medium to depths beneath the surface of the medium, the method comprising:
calculating the necessary operational parameters to generate the arbitrary plane of elevated temperature;
rotating two or more ultrasound sources about a rotational axis and linearly translating the one or more ultrasound sources while emitting ultrasound energy from the ultrasound sources,
one or more of the two or more ultrasound sources having a focal depth at a first depth within the range of depths and one or more of the two or more ultrasound sources having a focal depth at a second depth within the range of depths,
the first and second depth are different,
the rotating, the linearly translating, and the emitting being executed according to the calculated operational parameters.
